(19) United States
(12) Patent Application Publication
(10) Pub. No.: US 2023/0098643 A1
(43) Pub. Date: Mar. 30, 2023

(54) CORONARY OSTIAL CARDIAC CIRCULATION ISOLATION CATHETER AND VENTRICULAR UNLOADING REPERFUSION SYSTEMS AND METHODS

(71) Applicant: Rheoxtech LLC, Chicago, IL (US)

(72) Inventors: Neil J. Thomas, Chicago, IL (US); Gintaras A. Vaisnys, Chicago, IL (US)

(73) Assignee: Rheoxtech LLC, Chicago, IL (US)

(21) Appl. No.: 17/936,268

(22) Filed: Sep. 28, 2022

Related U.S. Application Data
(60) Provisional application No. 63/249,362, filed on Sep. 28, 2021.

(51) Int. Cl. A61M 25/10; A61M 25/00; A61M 5/142; A61M 5/168

(52) U.S. Cl. CPC A61M 25/1025; A61M 25/0082; A61M 5/142; A61M 5/16804

(57) ABSTRACT

An interventional ostium occlusion catheter includes a catheter tube with at least one port at a first end, a tip at a second end, and a central lumen, with a positioning component and an occlusion component on the surface of the catheter tube close to the tip. A method of coronary intervention using the catheter includes maneuvering the catheter to an ostium; inserting the catheter into a coronary vessel; activating the positioning component and the occlusion component; and delivering an infusion via the lumen. An interventional system for improving management of ischemic cardiac tissue during acute coronary syndromes includes the catheter; a blood infusion device that delivers controlled reperfusate; and a ventricular unloading device that manipulates myocardial oxygen demand in a coronary chamber. The operator can mitigate reperfusion-related and oxygen-related tissue injury by precisely modulating oxygen re-exposure when re-establishing flow and when using a ventricular unloading device.

### CROSS-REFERENCE TO RELATED APPLICATION

This application claims the benefit of priority of U.S. provisional application No. 63/249,362, filed Sep. 28, 2021, the contents of which are herein incorporated by reference.

### FIELD OF THE INVENTION

The present invention relates generally to catheters used in interventional procedures and, in particular, in interventional coronary procedures. More particularly, the present invention relates to methods and apparatus for isolating and controlling blood flow in the coronary arteries from the aorta. The invention also relates to the clinical arena of interventional cardiology and, in particular, the field of percutaneous coronary interventions and treatments for acute coronary syndrome (acute myocardial infarction and/or unstable angina). A method and apparatus are described that provide the operator with an ability to mitigate reperfusion-related and oxygen-related injury to a tissue by precisely modulating the level of oxygen re-exposure of the tissue prior to, during and directly after the time of an intervention to re-establish flow to an infarct vessel but also when a ventricular assist or support device is being utilized for ventricular unloading.

### BACKGROUND OF THE INVENTION

Considerable effort and resources have been devoted to reducing the burden of cardiovascular disease and, in particular, acute myocardial infarction which leads to, or contributes to, the development of heart failure throughout the world. Whether managed surgically or percutaneously, there has been a consistent decrement in mortality rate for the syndrome of acute myocardial infarction (AMI or acute MI) over time. The ability of cardiovascular specialists to favorably alter the natural history of acute MI, however, has been a pipeline in converting an epidemic of chronic congestive heart failure, which was previously a once highly fatal acute syndrome, into a chronic and costly illness for survivors in many cases and for the system and society at large. This epidemiological link between AMI and congestive heart failure with reduced ejection fraction (HFrEF) continues to burden the system. Because of the direct link between infarct size and unfavorable ventricular remodeling (tissue-level outcomes) and heart failure (a clinical outcome), minimizing ischemia and reperfusion-related injury during acute percutaneous coronary intervention (PCI) has become a high priority and an area of active investigation particularly as it relates to percutaneous LV unloading. See the following references, the contents of which are incorporated by reference.

Krumholz H M, Wang Y, Chen J, et al. Reduction in Acute Myocardial Infarction Mortality in the United States: Risk-Standardized Mortality Rates From 1995-2006 JAMA. 2009 Aug. 19; 302(7): 767-773).

Stone G W, Selker H P, Thiele H, et al. Relationship between infarct size and outcomes following primary PCI: patient level analysis from 10 randomized trial. J Am Coll Cardiol 2016; 67: 1674-1683.

Antman E M. Time is Muscle. J Am Coll Cardiol 2008; 52:1216-21

Bates E R. Achieving Aspirational Goals in Providing Primary Percutaneous Coronary Intervention Care. JACC. 2019 Nov. 25; 12(22):2269-2271.

Kapur N K, Alkhouli M A, DeMartini T J, et al.: Unloading the Left Ventricle Before Reperfusion in Patients with Anterior ST-Segment-Elevation Myocardial Infarction. Circulation. 2019; 139(3):337-346.

For patients who suffer from any form of acute coronary event, the heart muscle is deprived of adequate levels of oxygen for a variable period of time and along a range of severity until appropriate treatment can be initiated. In many cases, irreversible damage to the heart can result in infarction, with cell death occurring in one of more areas of the left ventricular or right ventricular myocardium or within the conduction system of the heart. In addition to the effects of this lack of available oxygen on cardiomyocytes and conduction tissue, it has become increasingly recognized that the endothelial cells lining the blood vessels (down to the capillary level) can also be damaged or can become impaired in their ability to allow flow or function even if the upstream infarct-related vessel is open and after flow is reestablished. This is known as “no-reflow’ or microvascular failure.

For patients with acute coronary thrombosis and infarction, established therapy is timely reperfusion of the culprit coronary artery by three main types of intervention: 1) thrombolysis (clot dissolving drugs), 2) opening the artery with a catheter and balloon, or 3) bypassing the artery (coronary artery bypass grafting [CABG] surgery) and restoring blood flow to the ischemic territory. Modern treatment of acute myocardial infarction (MI) or ongoing myocardial ischemia usually comprises performing balloon angioplasty with stent deployment, directional atherectomy with or without distal protection or even laser therapy and intracoronary declotting. Such procedures can all be broadly considered to be part of the clinical arena of percutaneous coronary intervention (PCI). Both percutaneous intervention and surgical bypass of the vessels to facilitate increased blood flow are performed to “rescue” myocardium or other cardiac tissue at risk from further damage by ongoing ischemia that may result in an extension of infarction or new areas of damage. During what could be defined as the “reperfusion era”, it has been observed that reestablishing proper flow into epicardial coronary arteries: (i) mitigates injury if it is performed in a timely fashion; and (ii) improves survival in large cohorts of patients presenting with the clinical syndrome of myocardial infarction. Simultaneously, however, it has been observed that in certain circumstances, especially in cases of protracted or severe ischemia, reintroduction of blood flow and oxygen can ramp up the injury in a manner consistent with what has been described as reperfusion injury.

In the last several decades, considerable effort has focused on limiting infarct size and other manifestations of post-ischemic injury. The main purpose is to limit the amount of heart muscle damaged and, thereby, limit unfavorable changes in cardiac function that are known to result in congestive heart failure. There is a definitive link from an epidemiological point of view between myocardial infarction and heart failure. The concepts of ischemic pre- and post-conditioning (manipulating blood supply either locally or remotely) suggest highly evolved mechanisms by which the heart can protect itself from ischemia under certain conditions. Long-standing investigation points to a number of molecules that bind to receptors on the cell membrane (ligands) that then act as mediators of an intracellular signaling pathway or transcriptionally mediated mechanisms that can mitigate injury. In addition, the last fifteen to twenty years has allowed for a broader understanding of the membrane-bound ionic pump disturbances that develop as ischemia progresses and the resultant ionic membrane shifts that are involved in the development of post-ischemic contracture when the affected tissue is re-exposed to blood containing oxygen. These disturbances may also point to mechanisms of conduction system dysfunction and/or post-ischemic arrhythmias.

Numerous methods of reducing ischemic insults to tissue, such as through interventional catheters that allow infusion of the patient's own oxygenated blood, have been contemplated. For example, U.S. Pat. No. 5,403,274 by Cannon provides an apparatus for passively perfusing blood past a stenosis using pressure equalization. U.S. Pat. Nos. 5,573,508 and 5,573,509 by Thornton, assigned to Advanced Cardiovascular Systems (ACS), are directed to an intravascular catheter with a perfusion lumen that can be expanded to increase the flow of oxygenated blood or other body fluids when the distal portion of the catheter is occluded. U.S. Pat. No. 5,308,356 by Blackshear provides a passive perfusion catheter with a balloon that defines at least one passage to permit blood flow when the balloon is pressed against the wall of the blood vessel. Similarly, U.S. Pat. No. 5,505,702 by Arney, assigned to Scimed Life Systems, provides a dilation catheter with a composite balloon that allows passive blood flow past the catheter during dilation. U.S. Pat. No. 5,344,402 provides a low-profile drug delivery catheter with at least one port to permit perfusion of the upstream blood while the drug delivery balloon is inflated. U.S. Pat. No. 6,302,865 provides a guidewire with a perfusion lumen allowing for perfusion of the arterial blood past an inflated balloon.

To increase blood flow and reduce ischemia, active perfusion catheters have also been provided that allow perfusion of high oxygen content fluids past an infarct area. U.S. Pat. No. 5,137,513 by McInnes, assigned to ACS, provides a catheter and method of ‘active’ perfusion, wherein oxygenated blood, preferably from the upstream artery is supplied during inflation of a balloon. Similarly, U.S. Pat. No. 5,807,331 by den Heiher and Solar, assigned to Cordis Corp., provides an active perfusion catheter where blood or other high oxygen content fluids are perfused past the obstruction during balloon inflation.

Higher oxygen replacement has also been contemplated. For example, European Patent No. 0836495 provides an apparatus for delivering oxygen-supersaturated solutions during clinical procedures such as angioplasty. Recent clinical trials on such systems have failed to show any significant benefit from the use of supersaturated oxygen therapy. Similarly, U.S. Pat. No. 6,454,997 by Divino et al., assigned to Therox, Inc. provides a high oxygen content fluid through a catheter in an attempt to reduce ischemic injury by combining an oxygen-supersaturated fluid with patient blood. U.S. Pat. No. 5,186,713 by Raible, assigned to Baxter International, Inc. provides a method and device for the flow of oxygenated perfusion fluid, preferably the patient's blood, by active perfusion through an oxygenator.

Although there have been significant advances in reducing ischemia, a major area of focus has become reducing or even preventing injury that occurs after a rapid return of normal blood flow. Typically, coronary intervention after acute MI involves percutaneous transluminal coronary angioplasty either with or without subsequent stent deployment. After a short episode of myocardial ischemia, reperfusion of the area with the patient's blood results in the rapid restoration of cellular metabolism and function. In clinical situations in which ischemia is more protracted or severe, even with the successful treatment of occluded vessels and stenting a serious risk of heart dysfunction and even death still exists. If the ischemic episode has been of sufficient severity or duration, reperfusion may, paradoxically, result in additional injury related to reperfusion and re-exposure to oxygen and, if severe, ultimately worsening of heart function by unfavorable molecular and transcriptionally mediated events known as “remodeling”.

Reperfusion injury occurs in tissue when blood supply returns to the tissue after a period of protracted ischemia. The absence of oxygen and nutrients from appropriate flow of blood, if prolonged, creates a condition in which the subsequent restoration of circulation in an uncontrolled manner results in: inflammation, attachment of adhesion molecules, transcription of genes that mediate additional (programmed) cell death (known as apoptosis), other forms of upregulated inflammatory events, the opening of mitochondrial transition pores, and oxidative damage through the induction of oxidative stress; rather than restoration of normal flow and function. This damage is distinct from the injury resulting from the ischemia per se. Reperfusion injury may be due in part to the inflammatory response of damaged tissues involving the production of reactive oxygen species, resulting in damage to lipid bilayer cellular membranes, endothelial cell dysfunction; micro-vascular injury, alterations in intracellular Ca²⁺, sodium, potassium and hydrogen ion homeostasis, changes in myocardial metabolism, and activation of neutrophils, platelets, and the complement system. In addition, white blood cells carried to the area by newly returning blood cause the release of a host of inflammatory cytokines and other factors such as interleukins as well as free radicals in response to tissue damage. Under certain conditions, therefore, the restoration of blood flow to ischemic tissue exposes the tissue to levels of oxygen that can be damaging.

Key calcium ion fluctuations or oscillations triggered by the presence of molecular oxygen that lead to various degrees of contracture can also occur. These consequences may be mitigated by avoiding exposure of the tissue at risk to hyperoxygenated (or even relatively hyperoxic) perfusates, which create large tissue oxygen gradients. High oxygen gradients have been observed to create a consistent pattern of injury during the initial phase of reperfusion/reoxygenation. Such injury can be prevented by limiting pO₂ of perfusate during the PCI as described herein. These events may explain why supersaturated oxygen therapy has not been impactful in improving outcomes in the analysis of most trials of such therapy.

It is known that reoxygenation injury can occur after reestablishing blood flow (perfusion) to a previously ischemic tissue. It has not been widely appreciated that the severity (intensity and duration) of the antecedent ischemic conditions sets the stage for significant oxygen-related damage depending upon certain conditions that exist at the moment flow is reestablished or ischemia is eliminated. Furthermore, it has not been appreciated that even a brief period of abrupt oxygen re-exposure to ischemic tissue can initiate damaging oxidative stress, result in numerous inflammatory and gene-related processes, and lead to increased injury due to ionic imbalances that develop during the ischemic period.

It is also not appreciated that, especially in the newly deployable therapies where oxygen demand is reduced through techniques such as ventricular unloading, reperfusion injury can occur even before flow is reestablished into the infarct artery when oxygen demand (O₂D) is lowered by reducing wall tension of the LV chamber, thus improving the supply/demand balance to mitigate ischemia but paradoxically simultaneously creating a vulnerability to an oxygen gradient present in the milieu at that moment. With significant enough reduction in wall stress (wall stress or tension and heart rate are the two largest components in determining oxygen demand in the heart) and therefore myocardial O₂D; ischemic conditions can be eliminated in areas surrounding the central infarct zone. Areas surrounding the infarct may be supplied by, for example, collateral vessels or as another example, ischemia induced alterations in micro-vascular blood flow that may be dependent upon nitric oxide or other mediators of localized vascular tone and flow. Collateral flow may be from the right coronary artery to the left or vice versa or through collaterals from the LAD to the Circumflex or vice versa and so on. These collaterals can be the source of just enough supply to cause reperfusion injury when the demand has been reduced sufficiently by ventricular unloading.

There remains an unmet need to provide a reliable method of preventing post-angioplasty reoxygenation injury, especially in case of the newly evolving therapies, which are being deployed utilizing the initiation of vented cardiopulmonary bypass (as in coronary bypass surgery or extracorporeal membrane oxygenation [ECMO]) or using ventricular unloading devices such as percutaneous ventricular assist devices (pVADs). These devices and techniques can manipulate tissue condition during PCI by manipulating oxygen demand, but without corresponding control of the oxygen supply side, inadvertent reperfusion and reoxygenation injury can result.

Additionally, in traditional cardiac surgery, it is routine practice to isolate the cardiac circulation from the systemic flow to the rest of the body. Compartmentalization of flow is routine during relatively simple or more complex operations on the heart and great vessels and is practiced routinely during surgical interventions. Compartmentalized flow or control of flow in one arterial system or capillary bed vs. another is less well appreciated in percutaneous therapies. More modern applications in the heart catheterization lab (e.g., pVADs) may manipulate tissue conditions in a way that creates an unintentional vulnerability to oxygen re-exposure.

In a typical cardiac surgery procedure, clamping of the ascending aorta and the commencement of extracorporeal bypass allows the surgeon to isolate and treat the heart separately from the rest of the body. Cardiopulmonary bypass utilizes an external blood oxygenation and pumping circuit and provides circulation to the main systemic blood circuit of the patient, excluding the heart's own blood circuit. This compartmentalization of flow allows arresting the heart and allows the surgeon a quiescent and mostly bloodless field.

Once the heart is taken “out of the circuit”, it can be arrested and appropriate procedures and therapies can be applied to the heart (e.g., coronary vessel bypass, heart valve replacement, etc.). However, open heart surgery is a very invasive operation and more and more cardiac interventions are now being performed by interventional cardiologists. These procedures include angioplasty and stent deployment in coronary vessels and even deployment of aortic valve replacements via catheter-based delivery systems, as well as mitral valve repair and replacement procedures. These procedures are all done interventionally without cardiopulmonary bypass and without opening the chest. While this has advantages, in acute coronary events it cannot leverage the surgical concepts of compartmentalization of flow nor, until now, of ventricular unloading. Advances in ways to better help resuscitate and protect the heart suffering from ischemic conditions (e.g., decreased oxygen supply and blood nutrients) have not yet been applied impactfully during interventional procedures.

Evidence of the benefits of control of the composition of the blood which the ischemic tissue sees immediately after its ischemic conditions have been relieved by reestablishment of blood flow is well-established in the cardiac surgery literature. Furthermore, there is evidence that reducing the oxygen tension (pO₂) of the blood which is reintroduced into ischemic tissue at the moment ischemic conditions are relieved is critical to mitigating reperfusion injury. This has been observed to be the case whether ischemia is eliminated by reduction of demand or improvement in supply or both. At this time, while interventional cardiologists have a tool to reduce LV wall tension and oxygen demand, they do not have the ability to control the coronary circulation independently from the systemic circulation and are not able to treat the ischemic heart in ways that may be significantly beneficial to the patient.

Ischemia arises from an imbalance between supply versus demand of oxygen in the blood (either too much demand or too little supply). A new paradigm recently being introduced into interventional cardiology procedures is the surgical concept of ventricular unloading. Vented cardiopulmonary bypass is known to reduce myocardial O₂D by fully fifty percent. While transvalvular pVADs are not quite as effective at mechanically unloading the chamber, they are being facilitated by newer percutaneous technologies. Such unloading of a ventricle can reduce or may eliminate ischemic conditions in some areas by affecting the heart's demand side of the equation and without immediately addressing the supply side. While there is impressive data in large animal studies that infarct size may be reduced by first installing a transvalvular pVAD and then opening the infarct artery, this has not yet been observed to occur in humans or in any clinical situation where conditions may be quite different and impacted by numerous other factors. Until new coronary reperfusion technology is designed and approved, the interventionalist will continue to be unable to independently control reperfusate composition or conditions and this is known to have significant negative effects in many cases.

Control of the perfusate condition and composition is known in the surgical literature to be critical in coronary reperfusion therapy and has been studied over many decades. Many publications on this topic can be found in the literature. However, it is also important to point out a large difference between the protection of the heart from imposed surgical ischemia (by reducing myocardial O₂D to nearly zero) and the rescue or resuscitation of the tissue from ischemia already present at the time of the therapy. That stated, the applicability of such control over an entire arterial system or tissue capillary bed (as might also apply to lower extremity ischemia or, perhaps, cerebral artery ischemia) can have significant benefits. Recent publications have documented worse outcomes in post-cardiac arrest patients exposed to hyperoxic conditions, while over-oxygenating the brain and cerebral circulation in ventilated stroke patients has also suggested higher mortality and worse outcomes.

Depicted in FIG. 1, the heart's 10 arteries originate as the right 20 and left 24 main coronary arteries, which branch from the ascending sinus portion of the aorta 18 just above the aortic valve 28 (coronary ostia; see FIG. 9). There are typically three cusps of the aortic valve 28: the non-coronary cusp 16, which contains no ostia, the right coronary cusp 12, which contains the ostium 22 of the right coronary artery 20, and the left coronary cusp 14, which contains the ostium 26 of the left (main) coronary artery 24 (assuming no congenital anomaly is present). FIG. 1 depicts an interior view of the right 22 ostium; the left ostium 26 is shown in phantom. The ostia 26, 22 of the left and right coronary arteries 24, 20 are located just above the aortic valve 28 (see also FIG. 9). Oxygenated blood is pumped into the sinus portion of the aorta 18 from the left ventricle chamber 30 (i.e., a coronary chamber); it then flows into the coronary artery ostia 22, 26 and then to the coronary arteries 20, 24.

In an interventional setting, the heart must continue to beat to supply blood flow to the body. If the coronary blood flow is isolated, care must be taken to maintain adequate blood flow in the coronary circuit. The complete isolation of a section of the heart's coronary circulation can in theory be achieved by occluding and taking over the circulation at the coronary ostium with a catheter, but this has not previously been done in interventional settings. In fact, interventional catheters have always purposely been designed to avoid such total occlusion of the coronary artery and to allow flow from the aorta around the catheter into the distal circulation at all times but very brief intervals, to accomplish balloon dilatation and balloon assisted stent deployment. An interventional catheter that could facilitate complete control of coronary flow and composition would have to occlude the ostium but also be large enough (or adjustable) to accommodate adequate blood flow through the catheter to that corresponding coronary artery through an infusion lumen. Such a catheter would have to function both as a guide catheter (with particular backward dislodgement protections) and the source of all coronary blood flow for the duration of the therapy. However, before now interventionalists have concerned themselves with maintaining (not blocking or taking over) coronary artery blood flow, unless such a blockage was present pathologically.

Interventionalists have therefore not yet fully realized the benefits that may derive from management of ischemic tissue conditions, by both decreasing demand with unloading and controlling flow and composition of perfusate, including oxygen tension, simultaneously through surgical control of coronary flow. The complete compartmentalization and control of coronary flow is a surgical concept that relates to cardioplegia delivery. The concept of cardioplegia (from the Greek . . . cardio=heart; plegia=paralysis) almost always implies a solution used by a surgeon (not an interventionalist) to initiate controlled cardiac arrest during surgery. Thus “cardioplegia” solutions have not so far been seen separately as a vehicle to apply other therapies to the coronary circulation and to control aspects of reperfusion that would not otherwise be controllable in an interventional setting. This has been unavailable to the field of percutaneous intervention until this point and the benefits of controlling oxygen re-exposure and other components, which include not only reperfusion injury prevention but also the potential addition of other ligands (molecules known to be protective in ischemic conditioning of the heart) or drugs to be added as adjuncts.

Meanwhile, ischemic conditioning is the concept whereby (by various signaling pathways) either local or remote protection is conveyed to the heart. In the past thirty years, the molecular signals have been elucidated in more meaningful ways. Molecules such as nitric oxide (NO), adenosine, and even insulin are known to convey protection to the heart from ischemic injury, each by a different mechanism. These approaches can be utilized and have been studied in the surgical environment but have not been directly deployable in the interventional setting. Where they have been tried, they are incomplete or less than optimally timed and have not been demonstrated to be useful.

More recently, as part of a system to resuscitate a struggling heart, the deployment of a percutaneously deployable left-ventricular (LV) unloading and circulatory support device has been used to help the function of a heart that might be functionally struggling from ongoing or pre-existing dysfunction or acute ischemia. LV support devices have historically been used in the interventional setting not to control or manipulate tissue condition but used more as a safety net to improve the outcome of interventions judged to be “high-risk” by other criteria (e.g., lack of functional reserve). Percutaneous support systems have been approved for use in shock situations and as adjuncts to treat patients with poor ventricular function that may represent end-stage pump failure or bridge-to-decision in clinical scenarios where recovery of native cardiac function cannot be immediately predicted.

Implantable LV devices have been used as bridge-to-transplant or “destination” therapy for end-stage heart failure. Percutaneous left ventricular assist devices (LVADs) have not been used for the purpose of or widely appreciated to cause a reduction in oxygen demand of the cardiac tissue. But, if the demand drops, so does the requirement for supply, so while ischemic conditions may dissipate as overall demand drops, the amount of blood flow and its relatively high oxygen content may paradoxically cause a vulnerability to hyperoxic damage during the transition period. As ischemic conditions dissipate as a result of the reduction in oxygen demand, any operator must be aware of the moment or period of vulnerability to an oxygen gradient this condition may engender is certain cases. Controlled flow into any and all collateral vessels and the main infarct territory may be critical in this moment. By being able to isolate the coronary circulation from the systemic flow, blood flow and oxygen could in theory be controlled during this critical period, but this has not previously been done and is the essence of the current invention.

This newer concept, not previously appreciated by those skilled in the art, must be effectively disseminated among or within the interventional community if mechanical unloading is to be used to reduce infarct size or “protect” the heart during PCI. By mechanically unloading the LV chamber, manipulation of myocardial oxygen demand enough to eliminate ischemic conditions in some areas can lead to unintentional injury rather than protection. The paradox lies in that the improvement in the supply-demand ratio (merely by lowering myocardial oxygen consumption/demand [mVO₂/O₂D]) creates a moment or period of vulnerability to oxygen over-exposure. Oxygen over-exposure in this circumstance is any significant gradient of perfusate to tissue oxygen tension (pO₂).

It is also envisioned that a sensor deployable with percutaneous technique via the vena caval system or the arterial system into the ventricular muscle could be used to sample tissue conditions. Such tissue conditions would be a direct measurement or some surrogate of degree of ischemia present in various segments of the heart. In one embodiment, a steerable sensor may be positioned within the heart muscle with echocardiographic or fluoroscopic guidance to detect redox potential, infrared saturation sensing, nano-technology sensors based upon molecular conformational change induced by light refraction, or some other chemical reaction that nano-sensing might facilitate.

Rheoxtech, LLC (Chicago Ill., the contemplated applicant hereunder) has developed prior techniques to mitigate reperfusion injury. Those techniques and associated systems and apparatus are disclosed in the following U.S. Patents (incorporated herein by reference): U.S. Pat. Nos. 9,775,940, 9,623,167, 9,504,779, 9,221,371, 9,084,856, 8,888,737, 8,709,343, 8,562,585, 8,178,041, 7,708,942, and 7,455,812. Prior disclosures discuss use of venous blood and mixtures with arterial blood, and catheters with perfusion lumens to deliver it. However, those prior disclosures do not contemplate intermixing those techniques with either ventricular unloading devices or interventional ostium-blocking balloon catheters.

### SUMMARY OF THE INVENTION

In one aspect of the present invention, an interventional ostium occlusion catheter comprises a catheter tube having at least one port at a first end, a tip at a second end opposite the first end, and a central lumen; a positioning means joined to a surface of the catheter tube distal from the port; and an occlusion means joined to the surface of the catheter tube distal from the port.

In another aspect of the present invention, a method of coronary intervention comprises providing an interventional ostium occlusion catheter having a lumen, a positioning component, and an occlusion component; maneuvering the interventional ostium occlusion catheter to a first coronary ostium; activating the positioning component; activating the occlusion component; and delivering an infusion via the lumen.

In another aspect of the present invention, a method of coronary intervention comprises providing an interventional ostium occlusion catheter having a lumen, a positioning component, and an occlusion component; maneuvering the interventional ostium occlusion catheter to a first coronary ostium; inserting the interventional ostium occlusion catheter into a first coronary vessel; activating the positioning component; activating the occlusion component; and delivering an infusion via the lumen, inserting an interventional catheter through the lumen and performing an interventional procedure.

In another aspect of the present invention, an interventional system for improving management of ischemic cardiac tissue during acute coronary syndromes comprises an ostium occlusion catheter; a blood infusion device; and a ventricular unloading device operative to manipulate myocardial oxygen demand in a coronary chamber, wherein the blood infusion device is operative to deliver controlled reperfusate.

These and other features, aspects and advantages of the present invention will become better understood with reference to the following drawings, description, and claims.

### BRIEF DESCRIPTION OF THE DRAWINGS

FIG. 1 is a perspective schematic view of an aorta, illustrating the position of main arteries and their ostia;

FIG. 2 is a schematic view of an occluding catheter according to an embodiment of the present invention;

FIG. 3 is another schematic view thereof, illustrating use with a reperfusate pump;

FIG. 4 is a detail schematic view thereof, shown in use;

FIG. 5 is a detail schematic view of an occluding catheter according to another embodiment of the present invention, shown in use;

FIG. 6 is a detail cross-sectional view of the occluding catheter of FIG. 2, illustrating channels within the catheter leading to each balloon;

FIG. 7 is another detail cross-sectional view thereof, illustrating a guidewire passing through the lumen;

FIG. 8 is another detail schematic view thereof, illustrating passage of a therapy catheter through the lumen of the occluding catheter;

FIG. 9 is a schematic view of a heart, illustrating use of two of the occluding catheters of FIG. 2 in combination with a percutaneous ventricular assist device; and

FIG. 10 is a schematic view of a method of perfusate treatment, illustrating passage of the catheter from the inferior vena cava to a coronary bed of a heart.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is an interventional ostium occlusion catheter comprising a catheter tube with a central lumen, a surface, a positioning means, and an occlusion or occluding means.

Unlike other coronary guide catheters that take pains to avoid complete occlusion of a coronary artery vessel (e.g., by including side holes to maintain some level of perfusion), this catheter specifically and purposefully fully occludes the target vessel and provides a means to take over perfusion of that vessel and the entire subtended tissue and capillary bed. This represents a deviation from what is currently practiced or understood to be safe by those skilled in the art. It also represents what is explained herein to be a convergence of the practices of interventional cardiology and cardiac surgical protection in an innovative and non-obvious way.

The positioning means is a positioning component operative to control how deep into the main artery the occluding means goes to avoid accidentally occluding a branch. In some embodiments, the positioning means is a positioning balloon configured to be larger than the ostium to be occluded and smaller than the inner diameter of the artery through which it is routed. In other embodiments, other means of maintaining positioning, such as tiny nitinol wire needles deployed to engage the ostium atraumatically or other mechanical means attached to the outer surface of the catheter, may be used.

In one embodiment, the catheter does not have a separate positioning means. Rather, the interventionalist carefully positions the catheter before inflating the occluding balloon, which serves both occlusion and positioning functions.

The occlusion means may be an occlusion component such as an occluding balloon. The occluding balloon must operate more gently than (for example) angioplasty balloons. Thus, the structure and material of the balloon, as well as the balloon inflation system, is such that careful control is possible to ensure only “soft” and “gentle” pressure against the arterial wall that completely occludes the vessel at the ostium.

A significant issue with conventional (i.e., currently commercially available) catheters is that when therapy catheters are deployed into areas that cause them resistance (e.g., a vessel occlusion), the force required to push the therapy catheter forward can cause a backwards force in the guide catheter which can dislodge it from where it was placed. Considerable effort has gone into addressing this need for backup support. The present invention mitigates this problem because the occluding balloon can provide considerable anchoring forces to counteract this effect.

The ostium occluding guide catheter may be introduced into the patient via an access vessel using conventional techniques, such as via femoral or upper extremity puncture. Given that the size of this catheter may be on the larger end of the range, it is most readily introduced through a femoral access route, although other points of introduction may also be used. It is envisioned in another embodiment that a shorter and larger or possibly “variable diameter” catheter may be used (since resistance to flow increases with decreasing radius and increasing length) and deployable via a surgical cutdown to the axillary-subclavian system (much nearer to the heart). The catheter may be guided over a wire to the root of the aorta using conventional fluoroscopy methods. It is maneuvered using conventional techniques.

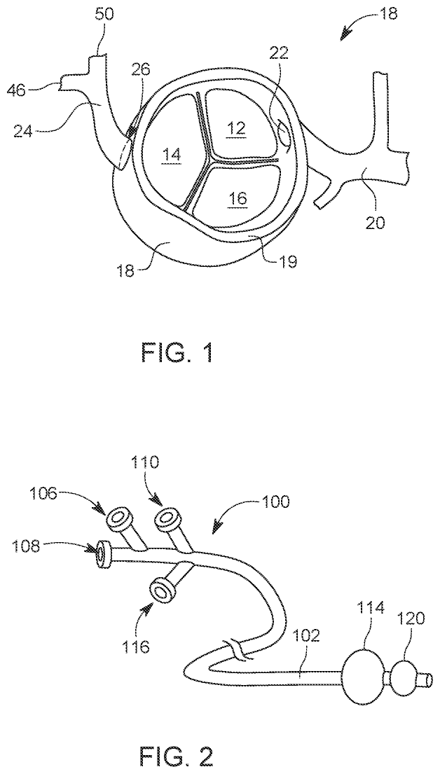

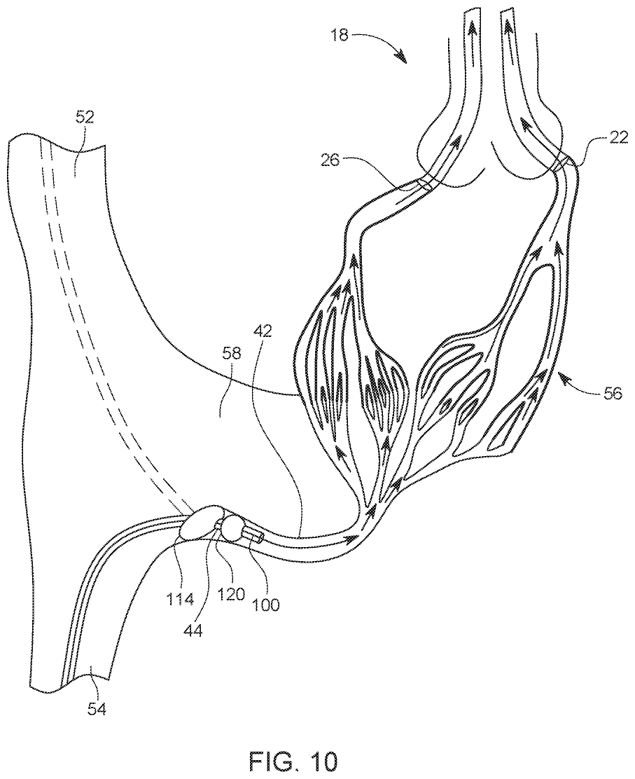

Catheters comprising features of the Ostium Occlusion Catheter (especially to create a “gentle” balloon occlusion) to enable the foregoing features and benefits can include modifications of those depicted in U.S. Pat. No. 10,980,652, FIGS. 1A and 1B; U.S. Pat. No. 7,862,601, FIGS. 1-3; and U.S. Pat. No. 10,092,429, FIGS. 10A-10B, 15-16, 18, and 42. Each of the named patents above is hereby incorporated in its entirety by reference, particularly for such catheter disclosures.

A method of use according to an embodiment of the present invention may comprise inserting the catheter and maneuvering the catheter into proximity to a coronary ostium. The positioning balloon is inflated to ensure that the catheter does not go too far into the coronary artery. The tip of the catheter may be allowed into the ostium before inflation to avoid having the aortic blood flow carry the catheter away. The catheter may be inserted into the ostium until the positioning balloon contacts the aortic wall and stops any further movement into the vessel. At that time, the occluding balloon may be inflated to fully occlude the vessel. The balloons are generally inflated using conventional techniques.

In some embodiments, the inventive method may provide deployment of two catheters [e.g., one for the right coronary artery (RCA) and one for the left main coronary artery (LM)] simultaneously. This allows the operator to control the entire coronary circulation in a manner similar to aortic clamping during cardiac surgery where the entire coronary circulation and the heart are isolated.

In cardiac applications, the catheter pair allows the coronary circulation to be isolated from the systemic circulation. This mimics the circulatory environment created in most open-heart surgery cases in terms of compartmentalization, but the heart is not arrested as with a “cardioplegia” solution. In this embodiment, the blood or solution delivered via the catheters can serve as a “vehicle” to deliver protection or adjuncts to protection either with or without simultaneous LV unloading and support. Each may also function as a guide catheter to support interventional procedures that include but are not limited to angioplasty and stent deployment.

Compartmentalization of flow and, potentially, segmentalized tissue condition determined with tissue condition sensors, is a critical component that can be used in conjunction with a number of other therapies, such as a percutaneous LV unloading device, coronary flow control, controlled oxygen exposure, and the addition of other molecules or ligands for protection that are known to be involved in protective signaling pathways. Thus, an interventional catheter (or catheters) allow(s) for the local circulation of the heart (or a different organ) to be taken over in part or in its entirety and controlled. These features and benefits have application not just to heart intervention therapies, but also the brain, other organs, and peripheral limbs. However, the heart must continue to work and supply sufficient blood and oxygen supply to the entire body during such procedures and while it is being intervened upon.

Although more comprehensive control of the cardiac circulatory system may be achieved utilizing a catheter per ostium, there are cases where deployment of a single catheter to control the circulation in one side of the heart can also provide significant therapeutic benefits. For example, a single ostium catheter may be used in a controlled reoxygenation procedure such as described in the Rheoxtech patents, discussed supra, during a coronary vessel angioplasty or other percutaneous intervention. The device herein disclosed, allowing blood flow through the lumen, may be advantageously used for such purposes.

In another embodiment, blood composition (including oxygen content) is controlled and varied over time during the procedure. Parameters including one or more of the blood composition, blood flowrate, pressure, temperature, pO₂, etc. may be controlled by the operator utilizing a blood infusion device, i.e., an external blood pumping mechanism, such as a reperfusate pump, and by adding or removing blood components or compounds (e.g., amount of oxygen, drugs, ligands known to impact signaling in protection etc.). External control of blood composition and dynamics may continue until the balloons are deflated and the catheter is removed. In some embodiments, a measurement of the conditions at the distal end of the catheter may be made and communicated to the pumping mechanism (e.g., pressure, oxygen content, etc.). This may be done by sensors at a distal end of the catheter or in some cases (e.g., pressure) by a channel in the catheter that communicates with a sensor at a proximal end of the catheter. Other types of tissue-based sensors are also envisioned.

Interventional devices such as therapy catheters may also be passed through the internal lumen of the catheter to perform cardiac interventions such as angioplasty or stent deployments. Having control of the blood composition at the moment therapy is initiated or, more particularly, at the moment ischemic conditions are relieved, is critical to minimizing reperfusion injury.

In some embodiments, prior to inflation of the balloon or deployment of other positioning means, blood flow may be established through the catheter lumen to ensure that, once partial or full occlusion is established, adequate blood flow is being provided to the coronary vessels. Blood flow may be provided by a blood pump such as a heart lung machine, an ECMO circuit, or a roller pump. Blood flow through the lumen may be maintained so long as the catheter is engaged within the ostium and until either balloon is deflated and the catheter is removed from the ostium. Preferably, blood pumping/flow is performed by structures, and blood content for the infusion is determined and mixed, as disclosed in previously discussed prior art.

In some embodiments, the main right coronary artery is engaged and/or the right ostium is engaged.

In some embodiments, the main left coronary artery is engaged and/or the left ostium is engaged.

While the previous discussion is specific to ostium occlusion during an interventional procedure on the coronary arteries (e.g., angioplasty, stent placement, occlusion removal), the foregoing techniques have application as well during pVAD usage (with or without simultaneous interventional procedures). An interventional system and method are described for improving the management of ischemic cardiac tissue during acute coronary syndromes. The system combines a catheter-based sub-system which allows for infusion of a carefully controlled perfusate during percutaneous coronary intervention, with or without simultaneous balloon dilation of a coronary artery, along with manipulation of the myocardial oxygen demand (O₂D) of the tissue by simultaneously implementing mechanical unloading of the chamber. In addition, systems and concepts are provided for modifying oxygen demand during an intervention that can be measured or monitored. This addresses the situation where the initiation of a pVAD that effectively unloads the heart at various flow rates, if ischemic conditions are relieved, paradoxically creates a moment of oxygen damage vulnerability for the previously ischemic tissue.

The system is described whereby as oxygen demand (O₂D) is reduced through mechanical unloading of the heart to a level that is satisfactory for elimination or significant mitigation of ischemia in some areas of the heart (which may suffer diffuse but heterogeneous ischemia), the conditions of flow into an “infarct artery” may be simultaneously controlled in a manner that provides protection from over-exposure to oxygen.

In one embodiment of the invention, a method of preventing reoxygenation injury during acute PCI is provided comprising the initiation of pVAD support that effectively and impactfully reduces myocardial oxygen demand (O₂D) and consumption (mVO₂). As O₂D drops, areas that become non-ischemic also become vulnerable to oxygen over-exposure. Control may be taken over the coronary circulation by occluding perfusion catheters and accompanied by immediate initiation of flow down the catheters to maintain antegrade blood flow and nutrient delivery. Although pVAD flow allows ongoing cardiac function, the workload of the heart may be reduced by as much as half and therefore careful control of the coronary circulation and blood composition is used to prevent reperfusion injury. The innovative therapy taught here is based at least in part on the discovery that the tissue is vulnerable to injury by merely lowering O₂D. As O₂D drops, the period of vulnerability to oxygen must be addressed in the moment ischemic conditions are eliminated, even though this occurs in a non-homogeneous manner.

In some embodiments, pVAD support is commenced prior to coronary blood flow being controlled. In other embodiments, coronary blood flow control is commenced prior to or during or at the same moment of initiation of pVAD support.

In some embodiments, the artery in the right coronary is engaged, and flow is started with controlled perfusate blood (such as Rheoxtech reperfusion disclosed in the Rheoxtech patents named above) into the artery in conjunction with the initiation of pVAD device flow, which is known to reduce O₂D and to improve microvascular collateral flow to the infarct area. In fact, most collateral coronary flow is not well represented by angiographic assessment. For that very reason, tissue rendered no longer ischemic is protected by control of oxygen tension of the perfusate in the collateral vessel flow.

In some embodiments, an artery in the left coronary circulation is engaged, and flow is started with controlled perfusate blood (such as Rheoxtech reperfusion disclosed in the Rheoxtech patents named above) into the artery in conjunction with the initiation of percutaneous ventricular assist device (pVAD) flow. As pVAD flow is started, ischemia may be relieved in the area at risk without the need for the infarct artery to be opened immediately. As such, flow into the arteries that may be contributing to the collateral circulation to the infract zone may be controlled. In one embodiment, the left main coronary artery is engaged, and in another, the left ostium is engaged.

In another embodiment, in conjunction with pVAD support of the heart, both coronary circulatory systems are engaged by perfusion catheters with an occluding member to seal and isolate the coronary blood flow allowing for the control of antegrade flow composition into the infarct artery and/or the other arteries for the purpose of enhancing protection and the avoidance of too much oxygen delivery and too-high an oxygen gradient at the moment oxygen demand drops with initiation of left ventricle (LV) unloading. In one embodiment the left main artery is engaged and/or the right main is engaged. In another embodiment, the left ostium and/or right ostium is engaged.

In some embodiments, once pVAD support and control of a circulatory system is established, the infarct artery may be opened to controlled flow as well (such as Rheoxtech reperfusion disclosed in the Rheoxtech patents named above).

In another embodiment, the coronary sinus is engaged at the moment ventricular unloading therapy is initiated with a pVAD. The coronary sinus ostium is engaged with an occluding perfusion catheter, and retrograde flow is initiated with controlled perfusate blood (such as Rheoxtech reperfusion disclosed in the Rheoxtech patents named above) from a catheter in the coronary sinus. As pVAD flow is started, ischemia may be relieved in the area at risk without the need for the infarct artery to be opened immediately. As such, retrograde coronary sinus flow may flow into the area at risk prior to reestablishing flow into the “infarct artery”. It may also flow into arterial territory contributing to the collateral circulation to the area at risk or the infarct zone and, in so doing, reperfusion conditions may be controlled.

In another embodiment, retrograde flow control is combined with a form of antegrade flow control; including creating a siphon for gentle run-off of retrograde flow, and so as not to be perfusing both antegrade and retrograde at the same moment. Simultaneous retrograde and antegrade flow is known to be safe, however, for brief periods of time particularly for deairing purposes.

Referring to FIGS. 1 through 10, FIG. 1 illustrates a sinus portion of an aorta 18 with ostia 22, 26 that may be occluded by the inventive occlusion catheter. The Figure is discussed in more detail in the Background.

FIG. 2 depicts an ostium occlusion catheter 100 according to an embodiment of the present invention, having a perfusion flow port 106, a therapy catheter port 108, a positioning balloon port 110, and an occluding balloon port 116 at a first end of a lumen 102 and having a positioning balloon 114 and an occluding balloon 120 at a second end of the lumen 102. While the balloons 114, 120 are shown spaced apart on the lumen 102, the balloons 114, 120 may overlap, may be adjacent to one another, or may be positioned with a gap therebetween.

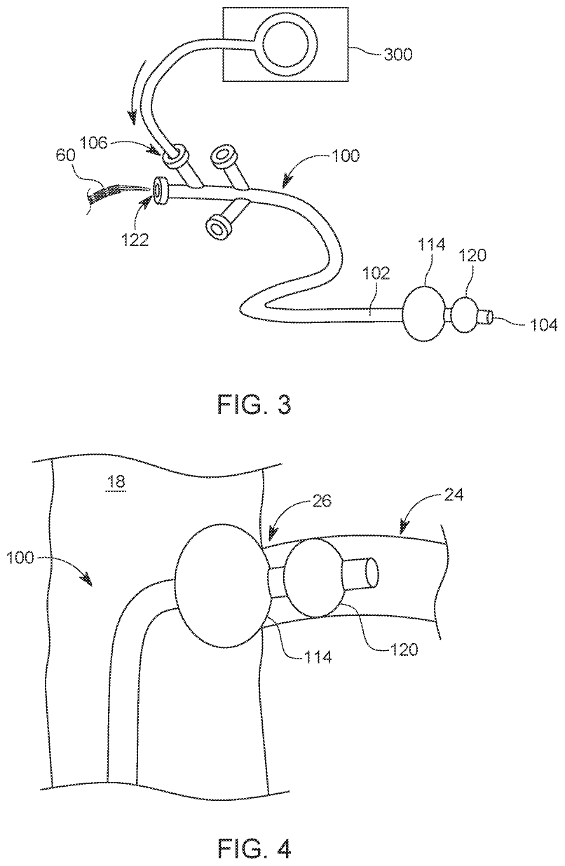

As shown in FIG. 3, an external blood pump 300, such as a reperfusate pump, fluidly communicating with the perfusion flow port 106 of the occlusion catheter 100 may be used in conjunction with the therapeutic techniques described above.

FIG. 4 illustrates an anchored catheter 100 engaged properly in the ostium 26 of the left main artery 24 from the sinus portion of the aorta 18. The blood composition, blood flow, pressure, temperature, pO₂, etc. may be controlled by the operator utilizing an external blood pumping mechanism, such as the reperfusate pump 300 shown in FIG. 3. In some embodiments, a measurement of the conditions at the distal end 104 of the catheter 100 may be made and communicated to the pumping mechanism 300 (e.g., pressure, oxygen content, etc.). This may be done by sensors (not shown) at the distal end 104 of the catheter 100 or in some cases (e.g., pressure) by a channel (such as those shown in FIG. 6) or separate channel providing fluid communication between the proximal end and the distal end (not shown) in the catheter 100 that communicates with a sensor (not shown) at the proximal end 122 (see FIG. 3) of the catheter 100.

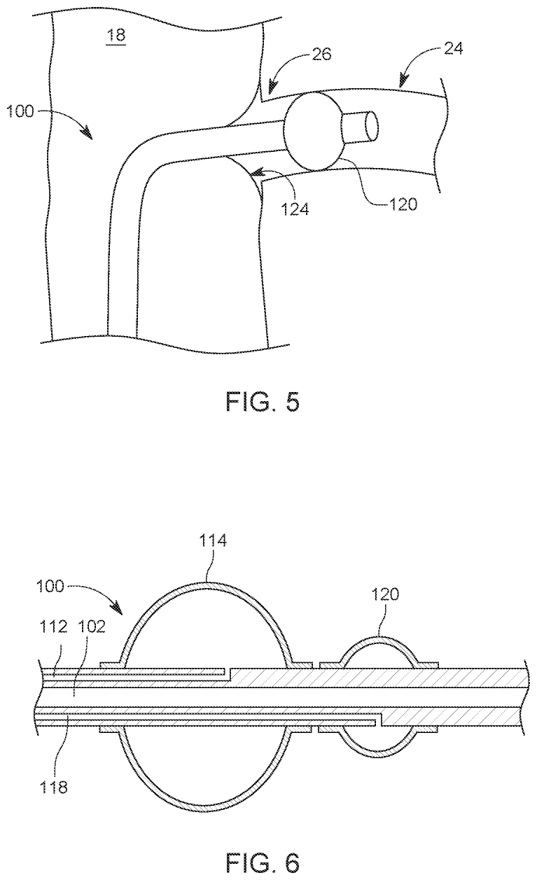

FIG. 5 depicts an alternative positioning means in the form of tiny nitinol wire needles 124 deployed to engage the ostium 26 atraumatically, so that the occluding balloon 120 may be inflated, occluding flow at a suitable location.

As seen in FIG. 6, the positioning balloon 114 fluidly communicates with the positioning balloon port 110 and is inflatable via a positioning balloon channel 112 and the occluding balloon 120 fluidly communicates with the occluding balloon port 116 and is inflatable via an occluding balloon channel 118. Both channels 112, 118 are isolated from each other and from other passages and lumens within the catheter 100.

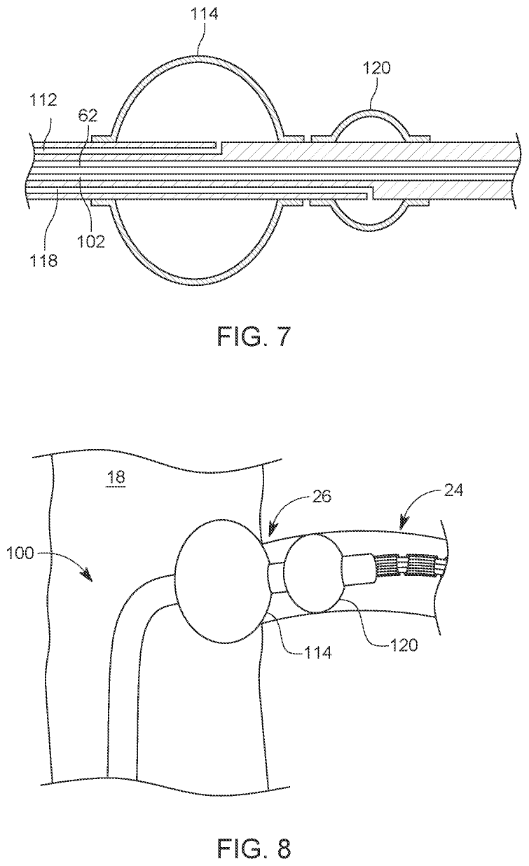

A guidewire 62 may be inserted through the lumen 102, as seen in FIG. 7. The catheter may be guided over the guidewire 62 to the targeted ostium.

FIG. 8 illustrates passage of an interventional therapy catheter 60 through the lumen 102 of the occluding catheter 100 according to an embodiment of the present invention.

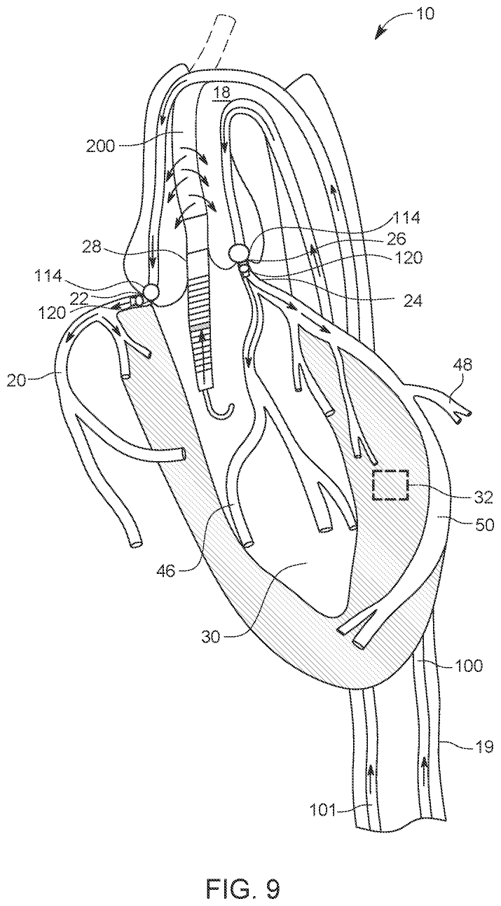

FIG. 9 depicts use of occluding perfusion catheters 100, 101 in conjunction with ventricular unloading therapy using a pVAD 200 according to an embodiment of the present invention. The pVAD 200 is inserted into the left ventricle chamber 30 through the aortic valve 28 by any suitable means. An inventive occluding catheter 100 enters the right main coronary artery 20 via the sinus portion of the aorta 18 and is positioned to occlude the right ostium 22. Another occluding catheter 101 enters the left main coronary artery 24 via the sinus portion of the aorta 18 and is positioned to occlude the left ostium 26 prior to the circumflex artery branch 46, the diagonal artery branch 48, and the left anterior descending artery 50. Arrows indicate the direction of flow, including within both catheters 100, 101 in the aorta 18 within the aortic wall 19. As pVAD 200 flow is started, ischemia may be relieved in the area at risk 32 within the ventricle muscle without the need for the infarct artery to be opened immediately. As such, retrograde coronary sinus 42 flow (see FIG. 10) may flow into the area at risk 32 prior to reestablishing flow into the “infarct artery”. It may also flow into arterial territory contributing to the collateral circulation to the area at risk 32 or the infarct zone and, in so doing, reperfusion conditions may be controlled.

FIG. 10 illustrates retrograde flow initiated with controlled perfusate blood from a catheter 100 in the coronary sinus 42 according to an embodiment of the present invention. As shown, the catheter 100 enters via the inferior vena cava 54 from the femoral arteries. However, deployment from the upper extremities (usually radial artery cannulation) via the superior vena cava 52 may alternatively be used, as illustrated in broken lines. The coronary sinus ostium 44 within the right atrium 58 is engaged with the occluding balloon 120 and positioned with the positioning balloon 114. Perfusate blood flow delivered to the coronary bed 56 is indicated by arrows. Note that the retrograde flow leaving the coronary bed 56 passes through the right and left ostia 22, 26 into the sinus portion of the aorta 18.

### EXAMPLES

An example of a method of practicing the inventions includes:

1) The pVAD and the occlusion catheters are advanced into position.

2) pVAD flow is initiated and as flow of the pVAD increases, a source of venous blood is taken and pumped antegrade through the occlusion catheters.

3) As O₂D drops and flow in collaterals becomes adequate to perfuse previously ischemic tissue, such collateral flow is controlled (for example, with a venous to arterial blood composition and pO₂ over a period of time).

4) After pVAD flow is initiated and controlled oxygen level flow into all collaterals has commenced immediately; PCI is performed next with simultaneous initiation of controlled flow into the infarct vessel.

5) After a period of time (for example, five to ten minutes of gradual reoxygenation), the catheters may be removed, and perfusion can be supplied via normal blood flow pathways.

6) pVAD flow continues as needed (for example, anywhere from thirty minutes to three days) depending upon the overall recovery of function of the heart.

7) If cardiac function is normal, pVAD flow may be weaned off after a period of time to ensure adequate percutaneous revascularization and controlled reperfusion and reoxygenation has been completed.

An example of a treatment process and flow to practice embodiments of the invention may include the following:

1. A patient with evolving infarction is brought to the interventional area.
a. The patient may have ST elevation MI (where it is assumed one artery or another is 100% occluded).
b. The patient may also have non-ST elevation MI or even unstable angina (both acute coronary syndromes) that harbor diffuse but heterogeneous ischemia.

2. In either case, the operator selects the artery to be opened first as dictated by the relevant clinical information.

3. Arterial and venous sheath access is obtained for blood pump flow and a site (or sites) for introduction of the interventional (PCI) catheters is chosen (likely femoral but may be an alternative site).

4. pVAD of whatever type is deployed.

5. Priority is given to engaging the coronary ostia with the occluding catheters and deployment of the pVAD into the ventricle for unloading rather than reestablishing flow into the infarct vessel.

6. pVAD is initiated with LV unloading commenced.

7. (pVAD flow may be, but is not likely, begun unless and until access to the coronary ostia is obtained and engaged.)
a. as LVAD flow is begun, wall tension drops and is governed by device flow rate.
b. as device flow is brought up; myocardial oxygen demand drops to a level that may eliminate ischemic conditions surrounding the central aspect of an infarction (in the case of a ST-elevation myocardial infarction [STEMI]) or may improve conditions diffusely but heterogeneously.
c. Paradoxically, this can create a moment of vulnerability of the ischemic but salvageable tissue to over exposure to oxygen.
d. Scientific research has shown and validated that at the moment of exposure of vulnerable tissue to an oxygen gradient, oxidative injury occurs and may be irreversible depending on the severity of the pre-existent ischemia.

8. In tissue sensors may be used to help inform and guide the process.
a. optionally, sensor technology is deployed to guide the starting point of the therapy and, possibly, its duration

9. Controlled reperfusate flow (venous pO2) is commenced immediately upon engaging the ostia and deployment of the occluding member of the catheter tip.

10. Alternately, coronary flow with controlled perfusate may be commenced before pVAD is deployed and/or pVAD flow is commenced.
a. As controlled flow into the coronary arteries is commenced and as pVAD flow is ramped up to unload the LV chamber, the myocardial oxygen demand drops by a variable amount that is dependent upon rate of flow of the device.
b. As device flow increases, wall stress (known to be a critical ingredient in what governs oxygen demand) drops.
c. Accordingly, mVO2 drops as well.
d. Certain coronary locations are at risk as demand drops
e. Ischemic conditions may in some cases be relieved merely by initiating device flow and, and in the border zone of the infarction, careful control of oxygen exposure is facilitated by the above-referenced techniques.
f. Especially in areas that may be reperfused by collateral flow previously embarrassed by either loading conditions, overall presence of multi-vessel coronary artery lesions, patient condition (hypertension, tachycardia from anxiety, etc.) or merely unable to keep up with demand prior to unloading (simultaneously): as demand drops, flow may improve.
g. The embarrassment and subsequent improvement in collateral flow is not necessary, however for the concept to be valid as micro-vascular flow is improved by decreases in wall tension in most cases.
h. Merely dropping demand (O₂D) may be enough to create this vulnerability to oxygen overload.

11. In this embodiment, the pO₂ of the controlled reperfusate catheter flow may be gradually ramped up over a period of (e.g., five to thirty) minutes (not hours) to effectively avoid any over-exposure.

12. In some cases, a percutaneously deployable sensor that samples various areas of the myocardium during the described resuscitation may be utilized.
a. TE echo may guide the deployment of the sensor into various areas with or without wall motion abnormalities to guide therapy.

It should be understood, of course, that the foregoing relates to exemplary embodiments of the invention and that modifications may be made without departing from the spirit and scope of the invention as set forth in the following claims.

What is claimed is:

1. An interventional ostium occlusion catheter comprising:
a catheter tube having at least one port at a first end, a tip at a second end opposite the first end, and a central lumen;
a positioning means joined to a surface of the catheter tube distal from the port; and
an occlusion means joined to the surface of the catheter tube distal from the at least one port.

2. The interventional ostium occlusion catheter of claim 1, wherein the at least one port is selected from the group consisting of: a perfusion flow port, a therapy catheter port, a positioning balloon port, an occluding balloon port, and any combination thereof.

3. The interventional ostium occlusion catheter of claim 1, wherein the positioning means is a positioning balloon configured to be smaller than an inner diameter of an access vessel and larger than an inner diameter of its ostium.

4. The interventional ostium occlusion catheter of claim 1, wherein the positioning means is nitinol wire needles configured to engage an ostium atraumatically.

5. The interventional ostium occlusion catheter of claim 1, wherein an occluding balloon is the positioning means and the occlusion means.

6. The interventional ostium occlusion catheter of claim 1, wherein the occlusion means is an occluding balloon.

7. A method of coronary intervention, comprising:
providing an interventional ostium occlusion catheter having a lumen, a positioning component, and an occlusion component;
maneuvering the interventional ostium occlusion catheter to a first coronary ostium;
activating the positioning component;
activating the occlusion component; and
delivering an infusion via the lumen.

8. The method of claim 7, further comprising inserting an interventional device through the lumen.

9. The method of claim 7, further comprising providing an external blood pumping mechanism operative to pump the infusion.

10. The method of claim 7, further comprising providing a ventricular unloading device operative to reduce oxygen demand in a coronary tissue.

11. The method of claim 7, wherein the infusion is delivered through the lumen prior to activating the positioning component.

12. The method of claim 7, wherein the first coronary ostium is a right ostium.

13. The method of claim 7, wherein the first coronary ostium is a left ostium.

14. The method of claim 7, further comprising inserting the interventional ostium occlusion catheter into a first coronary ostium before or after activating the positioning component.

15. The method of claim 7, wherein the first coronary ostium is a coronary sinus ostium, and the infusion is controlled perfusate blood; and further comprising initiating retrograde flow of the infusion via the lumen.

16. The method of claim 15, further comprising initiating antegrade flow control.

17. The method of claim 7, further comprising:
providing a second interventional ostium occlusion catheter having a lumen, a positioning component, and an occlusion component;
maneuvering the second interventional ostium occlusion catheter to a second coronary ostium opposite the first coronary ostium; and
activating a second occlusion component of the second interventional ostium occlusion catheter.

18. The method of claim 7, further comprising providing and initiating operation of a ventricular unloading device and delivering the infusion down the lumen of the interventional ostium occlusion catheter at a flowrate operative to maintain antegrade blood flow and nutrient delivery to the heart.

19. The method of claim 18, wherein operation of the ventricular unloading device is initiated before delivering the infusion.

20. The method of claim 18, further comprising opening an infarct artery.

21. The method of claim 7, further comprising:
controlling a blood parameter selected from the group consisting of: composition, oxygen content, flowrate, pressure, temperature, and any combination thereof.

22. The method of claim 21, further comprising measuring the parameter at a tip of the interventional ostium occlusion catheter.

23. The method of coronary intervention of claim 21, further comprising measuring the parameter utilizing a channel in the interventional ostium occlusion catheter.

24. An interventional system for improving management of ischemic cardiac tissue during acute coronary syndromes, comprising:
an occlusion catheter;
a blood infusion device; and
a ventricular unloading device operative to manipulate myocardial oxygen demand in a coronary chamber, wherein the blood infusion device is operative to deliver controlled reperfusate.

25. The interventional system of claim 24, further comprising an interventional therapy catheter within the occlusion catheter, operative to perform coronary intervention during take over of a coronary circulatory system.
